(19) United States
(12) Patent Application Publication
(10) Pub. No.: US 2008/0153084 A1
(43) Pub. Date: Jun. 26, 2008

(54) Obesity Markers And Uses Thereof

(75) Inventors: Louis Perusse, Sillery (CA); Luigi Bouchard, Quebec (CA)

Correspondence Address: BIRCH STEWART KOLASCH & BIRCH, PO BOX 747, FALLS CHURCH, VA, 22040-0747, US

(73) Assignee: Universite Laval, Quebec, QC (CA)

(21) Appl. No.: 10/565,847

(22) PCT Filed: Jul. 28, 2004
(86) PCT No.: PCT/CA04/01413

§ 371 Date: Feb. 15, 2007

Related U.S. Application Data
(60) Provisional application No. 60/490,535, filed on Jul. 29, 2003.

(51) Int. Cl. C12Q 1/68

(52) U.S. Cl. 435/6

(57) ABSTRACT

The present invention relates to a method for identification of a nucleotide substitution in the gene encoding for the neuromedin- or quantifying its levels of mRNA, to allow the determination of the susceptibility or predisposition of a patient to obesity, or to disinhibition or susceptibility to hunger. Determination of the susceptibility or predisposition to obesity or to hunger disinhibition can alternatively be determined by the identification of an amino acid.

### TECHNICAL FIELD OF THE INVENTION

The present invention relates to a method for determining susceptibility or predisposition of a patient to obesity comprising identifying in the patient an amino acid substitution in the neuromedin-β or a nucleotide substitution encoding gene thereof.

### BACKGROUND ART

Several problems of eating behavior have been described in the current literature since a long time ago. For example, anorexia nervosa (or nervous asitia, apocleisis) is known as a disease exhibiting psychotic symptoms such as a characteristic desire for emaciation and an abnormal eating behavior as well as somatic symptoms such as an extreme leptosome observed as a weight loss by 20% or more of the standard body weight as well as amenorrhea, and develops frequently in juvenile women.

In a current treatment, a less potent psychotropic agent or an anti-anxiety agent is administered depending on the symptoms and an oral tube feeding diet or a high calorie drip infusion is employed for recovery from an extreme physical exhaustion (See, “Today's treatment guideline”). However, no essential therapeutic agents capable of removing the psychotic symptoms characteristic of anorexia nervosa and also capable of normalizing the eating behavior have been reported.

Human growth hormone is still employed in the treatment of pituitary dwarfism and is believed to be effective also in the promotion of the healing of fractures and burn wounds and in the treatment of a patient having a reduced absorption of nutrition. Nevertheless, except for the improvement and exaltation in feeling associated with the recovery from a physical exhaustion state, no effectiveness of hGH against the typical psychotic symptoms has not been suggested.

PCT patent publication WO95/24919 disclosed that administration of hGH is useful against various diseases caused by the reduction in triiodothyronine (T3) which is a thyroid hormone. The inventors mentioned anorexia nervosa as an example of disease of T3 reduction syndrome, but all clinical effects of hGH they observed were an hGH-induced improvement in insufficient nutrition absorption only in the peripheral tissues after a trauma or an organ implantation, and all of their data (increased blood IGF-I level and reduced urinary nitrogen) can be interpreted based on the known peripheral effects of hGH.

Obesity is another major problem of eating behavior. It is now clear that public health is compromised by the obesity epidemic. This epidemic indicates that despite a better understanding of the obesity physiopathology and etiology our capacity to prevent weight gain and to treat obesity is far from good. Behaviors are important determinants of energy intake and expenditure and, to date, their role in the development of obesity was poorly investigated. Energy intake is modulated in part by food preferences and eating behaviors while physical activity behaviors may partly affect energy expenditure. Disequilibrium of these behaviors leads to energy homeostasis disturbance and to obesity when energy intake exceeds energy expense. Moreover, environmental modifications such as food abundance or settled way of life, which promote obesity, have changed at a very rapid rate since few decades.

Indeed, genetic, environment and their mutual interactions contribute to the modulation of these behaviors.

Three determinant factors assessed by the Three Factor Eating Questionnaires (Stunkard and Messick 1985, J. Psychosom. Res. 29:71-83) are used to define eating behavior and are identified as being cognitive dietary restraint, disinhibition and susceptibility to hunger.

Cognitive dietary restraint is a conscious behavior aimed at limiting food intake in order to control body weight, restraint lost is called disinhibition and susceptibility to hunger expresses the need of food perceived by the individual. Dysfunctional level of eating behaviors are a characteristic of obese people and are associated with obesity, weight gain and regain. Heritability of these traits was measured in two studies. In the Amish community, (Steinle, Hsueh et al. 2002, AM. J. Clin. Nutrition 75:1098-1106) reported heritability estimates of 0.28, 0.40 and 0.23 for cognitive dietary restraint, disinhibition and susceptibility to hunger respectively. In the Quebec Family Study (QFS) the heritability of disinhibition and susceptibility to hunger were estimated to be 0.19 and 0.32 respectively while the heritability of cognitive dietary restraint did not reach significance. Binge eating, bulimia nervosa and anorexia nervosa are characterized by dysfunctional cognitive dietary restraint, disinhibition and susceptibility to hunger levels compared to normal subjects' and have also an important heritability component. These heritability estimates favor the arguments that genetic is important in determining eating behaviors in individuals. However, this genetic component was poorly investigated.

It is interesting to observe that parenting styles (behaviors) may affect child development. It is known that parental feeding over-control is the best predictor of poor child self-control energy intake and is associated to greater child adiposity. To interpret this finding, the authors suggested that over-controlled child was unable to discriminate their internal hunger signal.

It would be highly desirable to be provided with new compounds and method to modulate the eating behavior. More again, it would be of particular interest to modulate the eating behavior through regulation of factors endogenous to human and animals.

### DISCLOSURE INVENTION

One aim of the present invention is to provide a method for determining susceptibility of a patient to obesity comprising identifying in the patient an amino acid substitution in the neuromedin β or a nucleotide substitution encoding gene thereof. The substitution is preferentially the replacement of a cytosine be an adenine at position 217 of exon 2 of the neuromedin β gene (c.217 C>A) (SEQ ID NO:1), corresponding to the replacement of a proline by a threonine at position 73 of the peptide sequence (p.P73T) (SEQ ID NO:2).

The method of the present invention is performed preferably for diagnosis of body fatness or abdominal/visceral obesity.

The susceptibility of a patient to obesity may also be representative of the disinhibition or susceptibility to hunger.

In accordance with the present invention there is provided a method for diagnosing predisposition or susceptibility to neuromedin-β associated eating behavior disorder comprising the steps of:

a) characterizing sequence or quantity of encoding nucleotide of neuromedin-β in a biologic sample of a patient; and

b) determining nucleic acid substitution or quantity in the characterized nucleotide sequence of step a);

wherein substitution of at least one nucleotide sequences in the nucleotide sequence, or its quantity such as in the case of MRNA quantification, is representative of the predisposition or susceptibility to obesity or a related or derivative disorder, or eating disorders. Still it will be understood by those skilled in the art that the nucleotide sequence can be as well a DNA

### BRIEF DESCRIPTION OF TIE DRAWINGS

FIG. 1 illustrates eating behaviors fine-mapping multipoint linkage analyses results for chromosome 15;

FIG. 2 illustrates the fat mass gain after six years follow-up for each neuromedin-β genotypes;

FIG. 3 illustrates the neuromedin-β protein sequences alignment of Homo sapiens (human) and Mus musculus (mouse);

FIG. 4 illustrates eating behaviors multipoint linkage analyses results for all chromosomes (chr);

FIG. 5 illustrates the genomic sequences of wild-type (from NCBI) and mutant Neuromedin β (NCBI and home-made sequencing) alignment.

FIG. 6 illustrates the gastric neuromedin β messenger RNA levels for each neuromedin-β genotypes; and

FIG. 7 illustrates neuromedin-β single nucleotide polymorphisms (SNPs) in accordance with the present invention.

### MODES OF CARRYING OUT THE INVENTION

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

In accordance with the present invention, there is provided a method and compound for modulating or controlling the eating behavior of humans and animals.

It has been discovered by the inventors that neuromedin-β and its related gene can be used to modulate eating behaviors of humans and animals that could have problems of weight control, or which would need a desired stimulation to increase or decrease food intake and growth at a certain level. In order to identify the neuromedin □gene, a genome wide scan linkage analysis was undertaken in the Québec Family Study. A locus affecting disinhibition and susceptibility to hunger was uncovered on chromosome 15q24.3. A fine mapping of this region led to the identification of the neuromedin β (NMB) gene.

Particularly, a non synonymous single nucleotide polymorphism located within the NMB exon 2 (SEQ ID NO:2) was found to be associated not only with disinhibition and susceptibility to hunger, but also with body fatness, body fat gain and macronutrient intake changes over time and gastric neuromedin-β messenger RNA (MRNA) levels.

Neuromedin-β belongs to the bombesin-like peptides that have been initially isolated from frog skin and later found to be widespread in mammalian neural and endocrine cells. In amphibians, where they seem to act as neuron-transmitters and/or neuromodulators, they have been classified into three subfamilies: the bombesins, the ranatensins, and the phyllolitorins. In mammals they modulate smooth-muscle contraction, exocrine and endocrine processes, metabolism and now behavior via binding to G-protein-coupled receptors.

In the present invention, different pharmaceutical formulation having a neuromedin-β activity may be employed. In view of the problems of the antigenicity, a mature neuromedin-β is preferred. Nevertheless, a purified product derived from a natural sources, having a stabilizing amino acid residue at the C- or N-terminal, and a recombinant neuromedin-β variant may also be encompassed in the present invention as far as they are the pharmaceutical formulations having neuromedin-β activities.

It will be understood that several types of pharmaceutical formulation capable of affecting or modulating the neuromedin activity via effects on its receptor are also included in the present invention. Alternatively, the formulations that can change the conformation of the neuromedin-β peptide may be combined therewith.

While the formulation may be a liquid formulation or a lyophilized formulation that can be administered by different ways, an oral formulation is preferred. Each of these formulations may contain a stabilizer and a carrier known in the art, and is used preferably as an isotonic solution or mixed to foods. The carrier may be a plasma-derived protein such as albumin, an amino acid such as glycine, or a saccharide such as mannitol. Generally, a lyophilized formulation for subcutaneous or intramuscular administration can also be employed.

The present invention will be more readily understood by referring to the following examples which are given to illustrate the invention rather than to limit its scope.

### EXAMPLE I

### Neuromedin β, a New Human Peptide Determining Obesity and Related Diseases

### Materials and Methods

The Québec Family Study (OFS)

QFS is a prospective family study on the genetics of obesity and its co-morbidities and was previously described (Bouchard 1996, In: Bray G, Ryan D, eds. Baton Rouge, La.: Louisiana State University Press ). This longitudinal study includes three phases: Phase 1: 1980-1982; Phase 2: 1989-1995 and Phase 3: 1997-2001. Anthropometric variables included weight (kg), body mass index (BMI; weight (kg) divided by squared height (m²)) and waist girth (WG, cm). Percent body fat (% FAT) was estimated from body density measurement obtained by underwater weighing (Behnke and Wilmore 1974, Englewood Cliffs, N.J.: Prentice-Hall.) and derived from the SIRI equation (Siri 1976, In: Lawrence J H, Tobias Calif., ed. NY, Academic press: pp. 239-280). Fat mass (FM (Kg)) was calculated from % FAT and body weight. A total of 660 subjects (≧217.5 years, 274 men and 386 women) from 202 families participating in QFS Phase 2 for which the TFEQ was completed were selected for linkage analysis and cross-sectional studies. The characteristics of these subjects are presented in Table 1. Prospective data from subjects participating in Phase 2 and 3 were available on a subsample of 295 subjetcs (136 men and 159 women). Food intake was measured with a 3-day dietary record as previously described (Tremblay, Sévigny et al. 1983, Nutr. Res. 3:819-830). In this example, the macronutrient intake was assessed as the percentage of the diet as well as to total energy intake.

TABLE 1
Characteristics of the subjects.

| | Men | Women |
|---|---|---|
| Number of subjects | 274 | 386 |
| Age (yrs) | 43.8 ± 15.1 | 41.9 ± 14.7 |
| Cognitive restraint | 5.9 ± 3.6 | 8.4 ± 4.8*** |
| Disinhibition | 4.5 ± 3.0 | 5.9 ± 3.4*** |
| Susceptibility to hunger | 4.1 ± 3.5 | 3.9 ± 3.2 |
| Weight (kg) | 85.5 ± 21.6 | 74.4 ± 22.6*** |
| BMI (kg/m²) | 28.6 ± 6.9 | 29.1 ± 8.8 |
| Waist girth (cm) | 96.8 ± 17.4 | 87.2 ± 19.0*** |
| Percent body Fat | 24.4 ± 9.4 | 33.2 ± 10.3*** |
| Fat mass (kg) | 22.3 ± 14.2 | 25.9 ± 15.5** |
| Total energy intake (kj)* | 11274 ± 3105 | 8532 ± 2131*** |
| Carbohydrate intake (%) | 47.2 ± 6.6 | 48.0 ± 6.3 |
| Protein intake (%) | 16.3 ± 3.0 | 16.4 ± 3.2 |
| Lipid intake (%) | 34.2 ± 6.1 | 34.1 ± 5.8 |

Data are means ± SD.
*= The number of subjects was 175 and 223 for men and women respectively.
**p ≦ 0.01 and
***p ≦ 0.0001.
Legend:
kg = kilograms,
m² = squared meter,
cm = centimeter,
kj = kilojoules.

### Eating Behaviors Measurements

The participants enrolled in this study completed the TFEQ (Stunkard and Messick 1985, J. Psychosom. Res. 29:71-83) as validated for the French population, (Luch 1995, in Nancy I, Université Poincaré:France). The fifty-one items of the TFEQ are scored as 0 or 1. The sum of points on 51 items were then aggregated into three scales: 1. Cognitive dietary restraint 2. Disinhibition 3. Susceptibility to hunger. Previous studies have shown that the TFEQ has good reliability and validity (Stunkard and Messick 1985, J. Psychosom. Res. 29:71-83); Laessle, Tuschl et al. 1989, J Abnorm Psychol. 98:504-507).

### Linkage Analysis Genotyping

Genomic DNA was prepared from permanent lymphoblastoid cells by the proteinase K digestion and QUIAGEN™ Blood & Cell Culture DNA Maxi Kit (Cat. No. 13362). Details on DNA preparation, polymerase chain reaction conditions and genotyping were described elsewhere (Chagnon, Borecki et al. 2000, Metabolism 49:203-207). Briefly, a total of 443 microsatellites and RFLPs markers spanning the 22 autosomes were selected from different sources, but mainly from the Marshfield Institute panel version 8a, were available for this genome scan. Markers map locations (in megabases (Mb)) were taken from the Human Genome NCBI resources (Built 31; http://www.ncbi.nlm.nih.gov/genome/guide/human/). The average intermarkers distance was 6.8 Mb ranging from 0 to 32 Mb. The highest and the lowest marker density was on chromosome 20 (3 Mb) and 21 (12 Mb) respectively.

### Neuromedin β Polymorphism (c.217 C>A or p.P73T) Genotyping

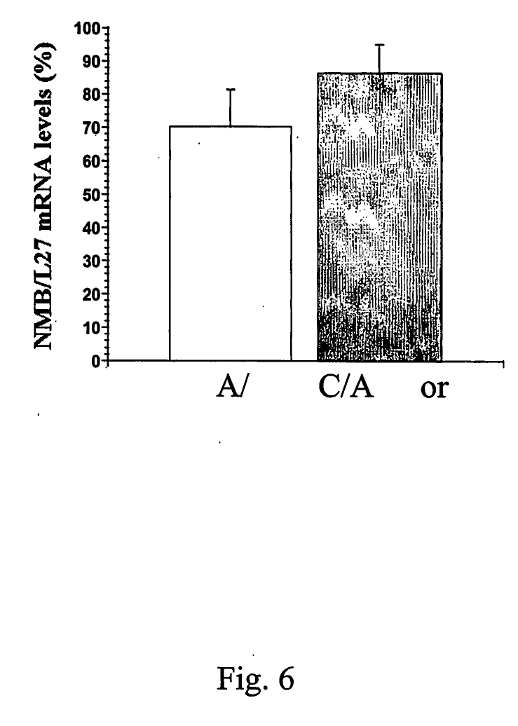

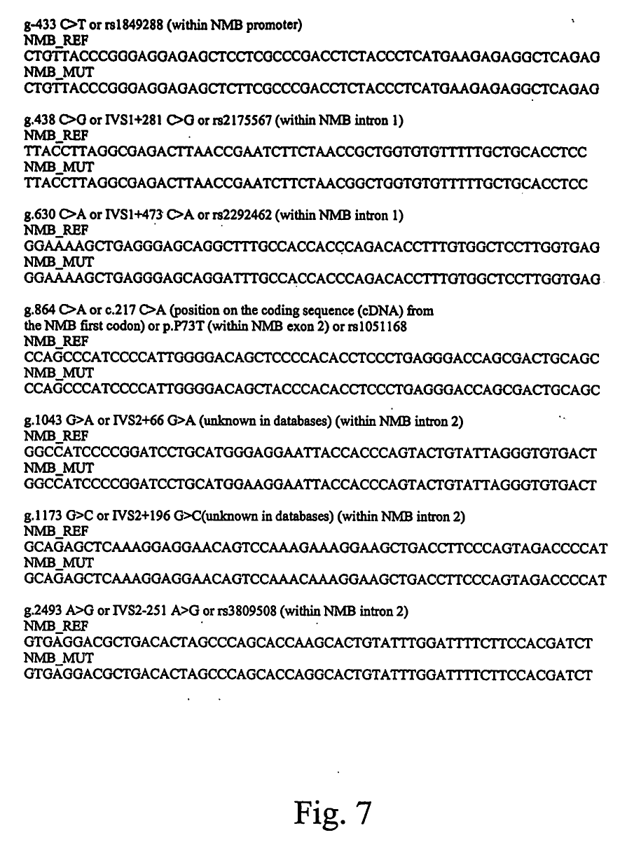

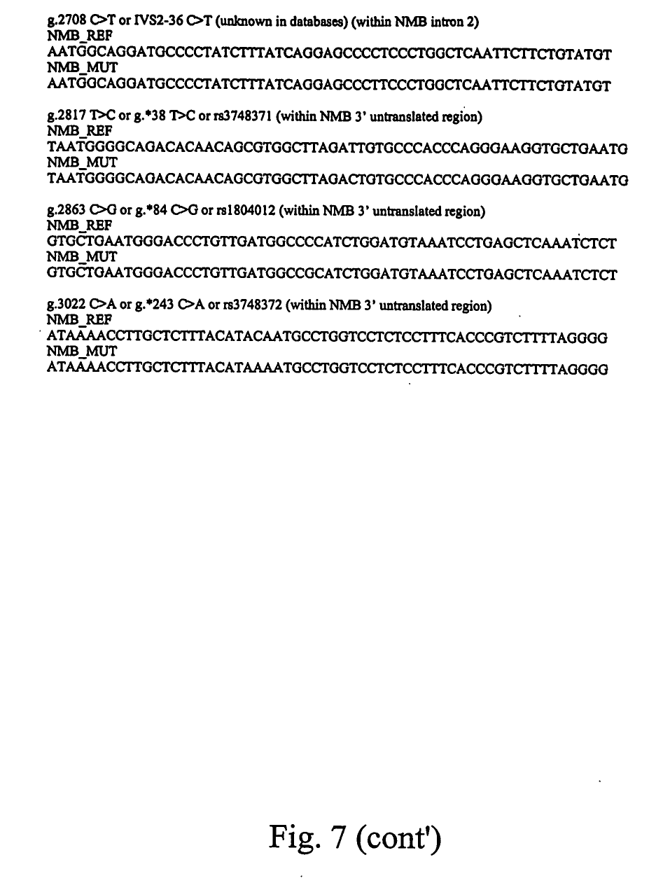

The use of c.217 C>A or p.P73T nomenclature design the same polymorphism on the coding sequence (DNA or ARN) and the peptide sequence respectively and are interchangeable (FIG. 6). On the coding sequence a C at position 217 is translated by a P (proline; genetic code for a proline: CCC) at position 73 on the peptide sequence. Alternatively, an A at position 217 on the coding sequence is translated by a T (threonine; genetic code for a threonine: ACC) at position 73 on the peptide sequence. In the genetic code between parentheses, the letter in-bold type and in italic design the mutated nucleotide of c.217 C>A. However, the c.217 C>A nomenclature will be mostly favored to facilitate the comprehension of this document.

### PCR Reaction

In a final volume of 6 μl, 20 ng of genomic DNA were added to a mixture containing a final concentration of dNTP (Amersham Pharmacia Biotech Inc.), 30 μM each; Taq DNA polymerase (QUIAGEN™), 0.3 U; buffer 1× (10×: TRIS-HCl, KCL, (NH₄)₂SO₄ and 15 mM MgCl₂; pH 8.7 (20° C.)); flanking primers, 50 nM each. Following a 5-min denaturation step at 95° C., 30 PCR amplification cycles were performed as follows: denaturation at 95° C., 20 sec; annealing 57° C., 1 min; for 10 cycles and denaturation at 95° C., 20 sec; annealing at 52° C., 1 min; for the remaining 20 cycles. In the same well, the PCR mixture dNTP's were digested using Shrimp Alkaline Phosphatase (USB), 0.2 U (final volume: 11 μl) for 15 min at 37° C. followed by 20 min at 80° C. Mini-sequencing assay, based on research done by Sun et al (Sun, Ding et al. 2000, Nuc. Acids Res. 28:E68), was performed in a final volume of 16 μl (in the same well); dTTP/ddNTP mix (dTTP, ddATP, ddCTP and ddGTP) (dNTP and ddNTP are from Amersham Pharmacia Biotech Inc.), 1.56 μM each; IRDye tag primers, 3.125 nM (LICOR); Thermosequenase (USB), 0,3 U; 0.6× buffer (10×: Tris-HCl, 260 mM, MgC12, 65 mM, pH 9.5) were added to microplates. Following 2 min denaturation step at 95° C., 30 PCR amplification cycles were performed as follows: denaturation at 95° C., 10 sec; annealing at 55° C., 30 sec; extension at 72° C., 5 sec. Detection was done on a LICOR automated sequencer model 4200.

### PCR and Mini-Sequencing Primers for c.217 C>A (p.P73T) Polymorphism Genotyping

PCR primers (forward (f), reverse (r)) and minisequencinig (ms) primers were as follows: f-5′-TGCAGTCGCTGGTCCCTC-3′ (SEQ ID NO:3), r-5′-AGGCGAGACTTAACCGAATC-3′(SEQ ID NO:4), ms-5′-CCTCAGGGAGGTGTGGG-3′(SEQ ID NO5).

### Statistical Analysis

Eating behaviors were adjusted for age and gender effects as well as for age, gender and BMI. These adjustments were performed separately in males and females using stepwise regression procedures. The residuals used in linkage analyses were standardized to a mean of zero and a SD of one.

Two approaches were performed to search for linkage between eating behaviors and the genetic markers. First, linkage was tested using the new Haseman-Elston regression-based method. The maximum number of sibpairs was 315. Linkage was tested using the SIBPAL2 software from the S.A.G.E. 4.0 statistical package (S.A.G.E. Statistical Analysis for Genetic Epidemiology 1999). Second, linkage was tested using the variance components-based approach implemented in the quantitative transmission disequilibrium test (QTDT) computer software (Abecasis, Cardon et al. 2000, Am. J. Hum. Genet. 66:279-292). Linkage analyses procedures were detailed elsewhere (Bosse, Perusse et al. 2003, Circulation 107:2361-2368).

A chi-squared (χ²) test was applied to evaluate if genotype and allele frequencies were in Hardy-Weinberg frequencies equilibrium and to compare genotypic frequencies between low and high scores of eating behaviors.

Men were less disinhibited than women and, for this reason, assignment to group of low or high disinhibition were performed for each gender separately. In men, cutoff values were 3 and 8 (0-3, 4-7 and 8-16), while in women the corresponding cutoffs were 4 and 10 (0-4, 5-9 and 10-16). For susceptibility to hunger, there where no gender differences and group assignments were the same in men and women with cutoff values of 2 and 6 (0-2, 3-6 and 4-14). Genotypic frequency differences were only tested between low and high groups of disinhibition and susceptibility to hunger.

Analysis of covariance was applied to compare mean values across genotypes. For cross-sectional studies, variables were adjusted for age and gender with or without further adjustment for BMI effects. For prospective studies, variables were adjusted for age, gender at phase 2 and delta age effects with or without further adjustment for baseline BMI effect. Deltas were obtained by subtracting Phase 2 to Phase 3 values. The mean follow-up was 6.0±0.9 years. Relatedness between family members was adjusted using the sandwich estimator and implemented in the SAS mixed procedure (Rice, Pérusse et al. 2002, Handbook of nutrition and food, C. D. Bernanier. Boca Raton, CrC Press, pp. 603-609). Mathematic transformations were applied to non-normally distributed variables (when needed). Reported Is means and standard errors are for untransformed variables and p-values are for transformed-one (when needed). Adjustment of the phenotypes for linkage analyses and other statistics (excluding linkage analyses) were performed using SAS software (version 8.02).

Neuromedin β and L27 genes MRNA quantification.

In order to evaluated the gastric neuromedin β content by real-time PCR, 10 gastric tissues from each NMB c.217 C>A or p.P73T genotypes (total n=30) were randomly selected from a tissue bank of morbidly obese individuals. The real-time PCR conditions were as follow: In final volume of 20 μl, 4 μl of cDNA were added to a mixture containing a final concentration of DNTP (Amersham Pharmacia Biotech Inc.), 50 μM each; Platinum Taq DNA Polymarase (Invitrogen), 0.5 U; Platinum Taq DNA Polymarase buffer 1× for NMB (20 mM Tris-HCl, 50 mM KCl (pH 8.4)) or homemade buffer for L27 1× (10 mM Tris-HCl; KCl 50 mM; 1 MM MgCl₂; and 0.15% Triton-X™); neuromedin β flanking primers (forward 5′-TTCCAGCCCATCCCCATTG-3′(SEQ ID NO:6) and reverse 5′-CAACAGGGAAGCAGGAAATAC-3′) (SEQ ID NO:7(SEQ ID NO: or L27 flanking primers (forward 5′-GGGCAAGTTCATGAAACCTG-3′(SEQ ID NO:8) and reverse 5′-CCTTGTGGGCATTAGGTGAT-3′) (SEQ ID NO:9), 250 nM each; 3 MnM MgCl₂ for NMB and 1 mM MgCl₂ for L27; and SYBR Green I (Molecular Probe) 1/25000. Forty five PCR amplification cycles were performed as follows: denaturation at 94° C., 15 sec; annealing 62° C., 30 sec and extension at 72° C., 15 sec for NMB and denaturation at 94° C., 20 sec; annealing at 62° C., 20 sec and extension at 72° C., 20 sec for L27. Quantification was done on a Rotor Gene 3000 (Corbett Research). Expression results were expressed as percentage of neuromedin P/L27.

### Results

### Genome-Wide Scan

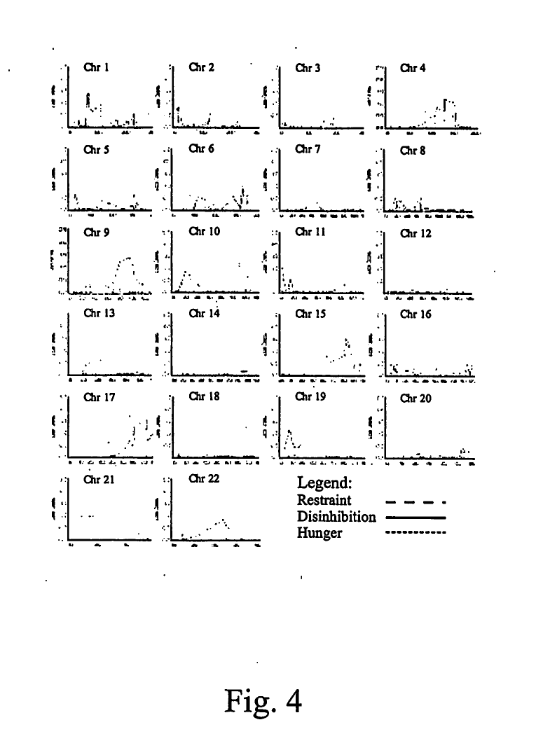

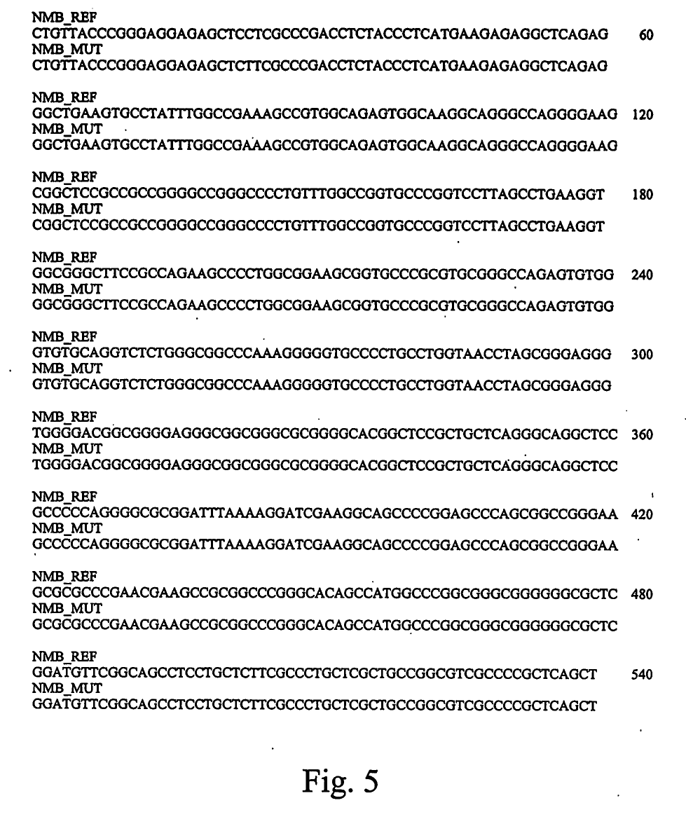

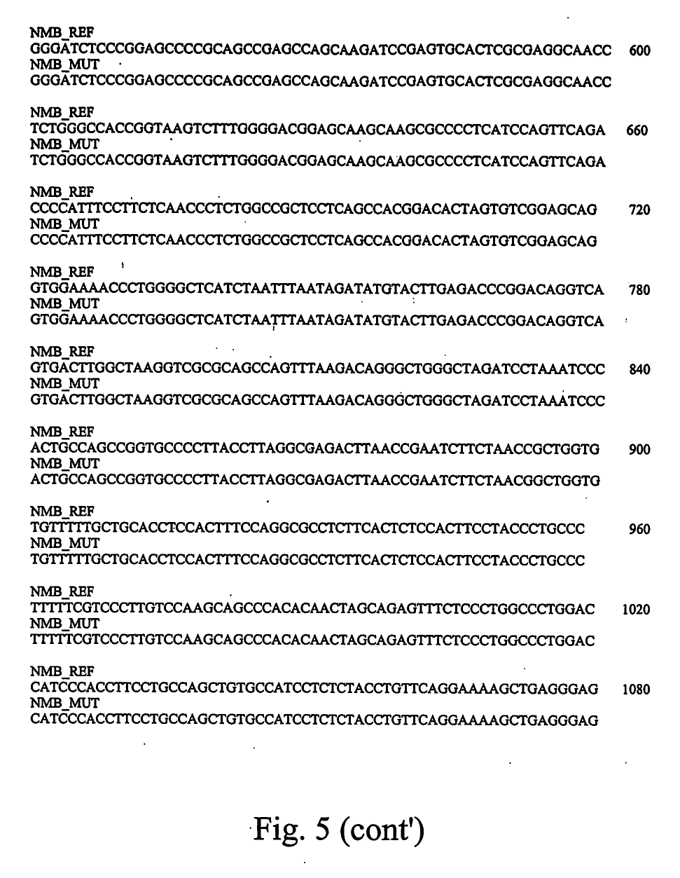

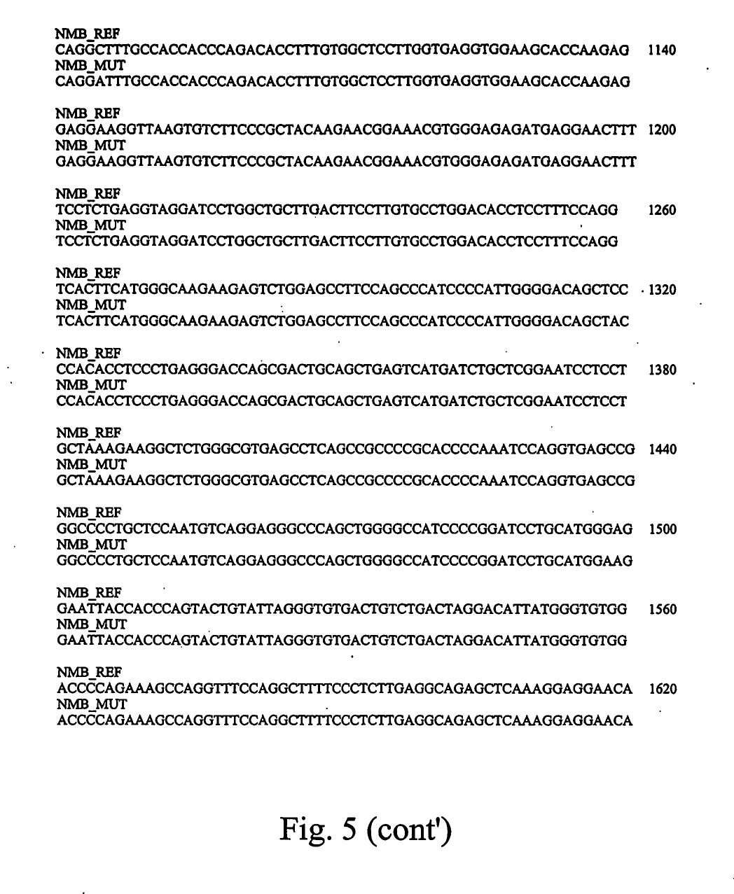

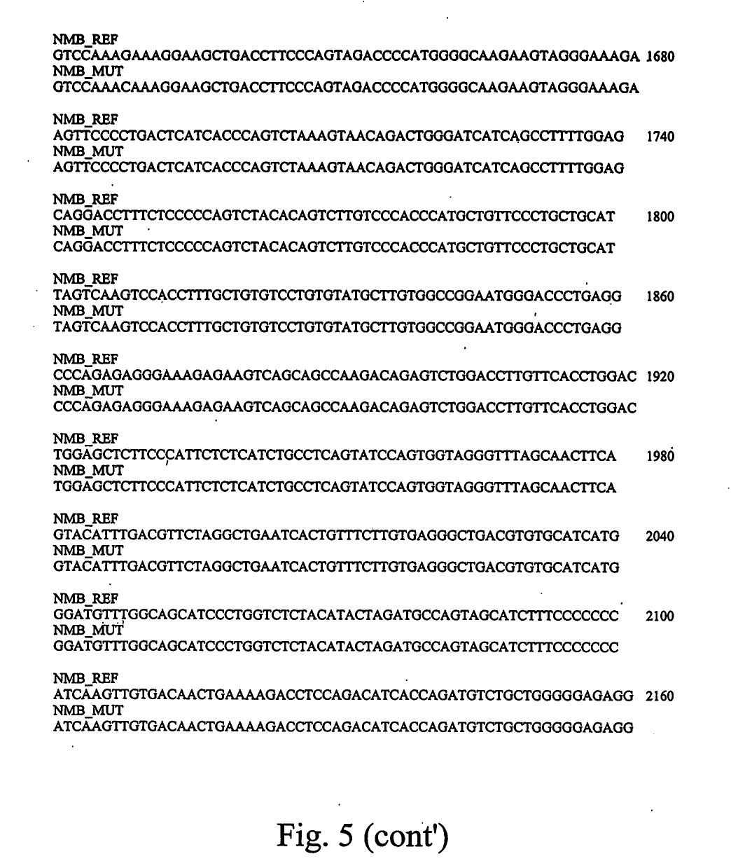

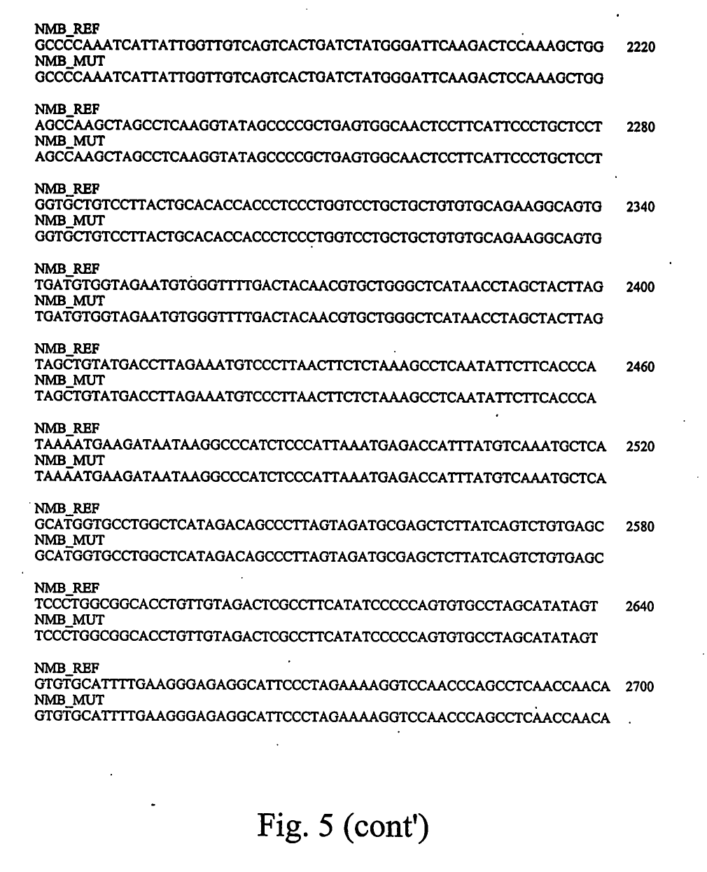

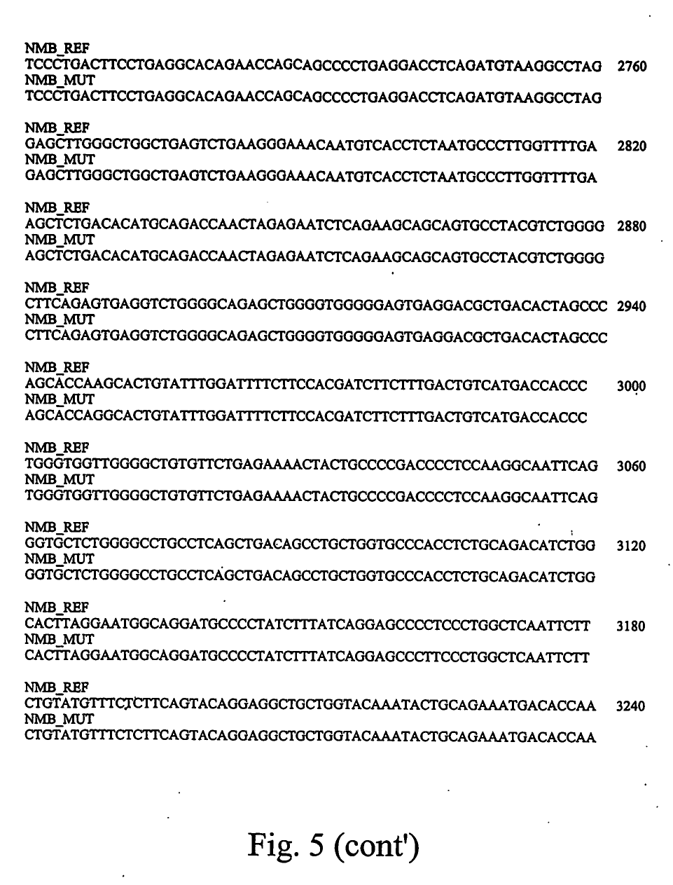

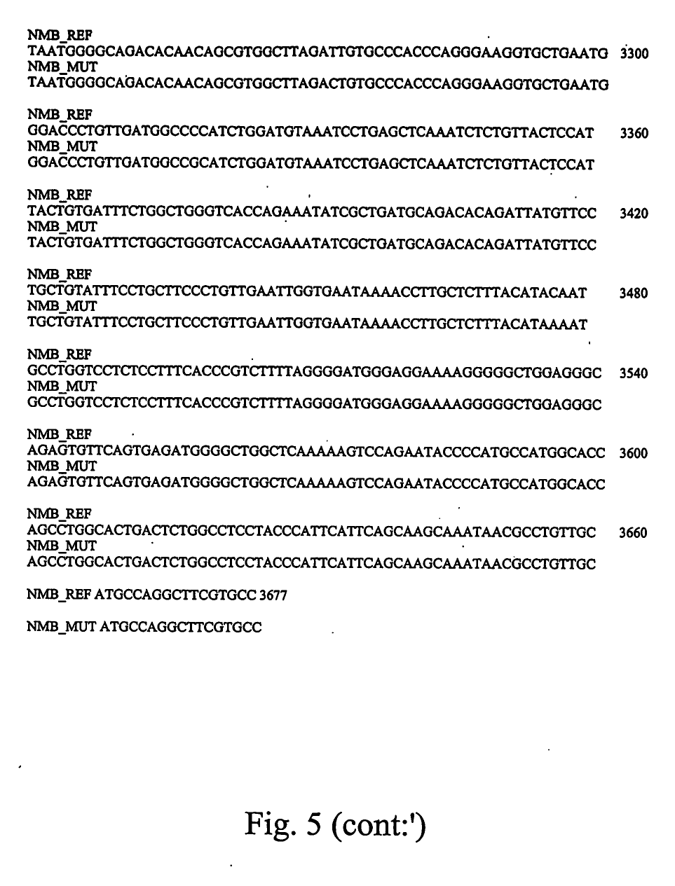

The complete eating behavior multipoint linkage analyses results are shown in FIG. 4 and Table 2 presents a summary of loci showing suggestive (p<0.01 and/or LOD>1.17) and promising (p<0.0023 and/or LOD>1.75) evidence of linkage based on at least one linkage methods. Briefly, five suggestive and promising evidences of linkage were found for disinhibition (1p31, 9q22, 15q24-q25, 17q23-q24 and 19p13) and six were found for susceptibility. to hunger (5q31, 13q32, 15q21, 15q24-q25, 17q23-q24 and 21q11). No significant linkage was found for cognitive restraint. Loci provided promising evidence of linkage are indicated in bold in Table 2. For disinhibition, promising evidence of linkage was found on chromosome 19p13 with marker D19S215 p0.002 (LOD=1.8); LOD=0.61). Three promising linkages were identified for susceptibility to hunger. These linkages were on chromosomes 15q21 with marker LHLNAIII (p=0.002 (LOD=1.76); LOD=1.03), 15q24-q25 with marker D15S206 (p=10.0001 (LOD=3.0); LOD=1.44), and 17q23-q24 with markers D17S1306 (0.006 (LOD=1.36); LOD=2.06), D17S1290 (p=0.007 (LOD=1.30); LOD=2.45) and D17S1351 (p=0.002 (LOD=1.74); LOD=0.95). Interestingly, the QTLs for susceptibility to hunger on chromosomes 15 and 17 were the same of those found for disinhibition and were not affected by BMI adjustments.

### Fine-Mapping

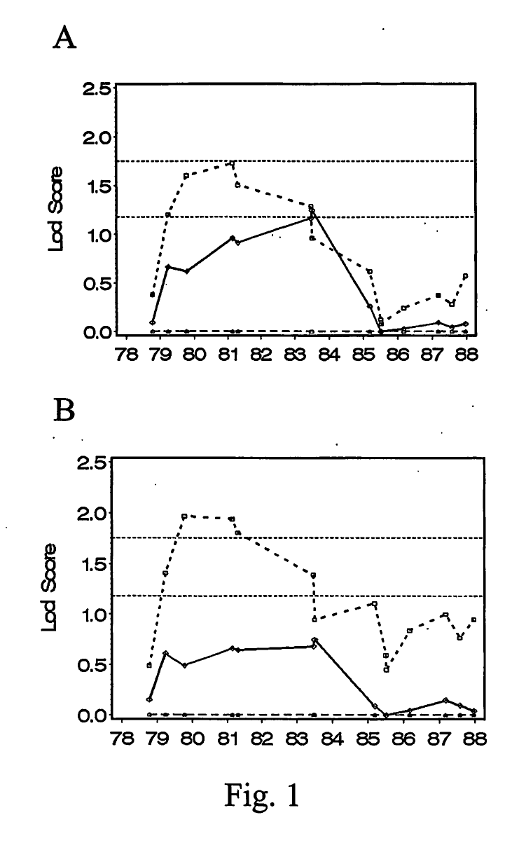

Fine mapping of the QTLs found for disinhibition and susceptibility to hunger on chromosomes 15 was then performed in order to diminish the span, in Mb, of this locus. Indeed, 10 additional genetic markers were genotyped (FIG. 1). For disinhibition, the locus found on chromosome 15 remained close to significant but did not reach significant level after BMI adjustment. The QTL found for susceptibility to hunger on chromosomes 15 remained significant after fine mapping even after BMI adjustment. For both phenotypes, the strongest evidence of linkage was found near D15S201 marker at 78.6 Mb.

### Association Studies

Association between one single nucleotide polymorphism (SNP), c.217 C>A (p.P73T), within the exon 2 of neuromedin β gene and eating behaviors was tested. Neuromedin β gene is located on the long arm of chromosome 15, at 78.2 Mb, near the suggestive evidence of linkage obtained with D15S201 for disinhibition and susceptibility to hunger.

For eating behaviors-related phenotypes, significant association was found between this mutation and disinhibition (p=0.0265, p=0.0057) and susceptibility to hunger (p=0.0343, p=0.0345) whether or not adjustment for BMI was made (Table 3). The portion of the variance attributable to c.217 C>A is 1.4% for disinhibition and 1.7% for susceptibility to hunger. For these associations, the A homozygous subjects had higher scores of disinhibition and susceptibility to hunger compared to the C carriers. No significant association was found between c.217 C>A and cognitive dietary restraint (Table 3). Cross-sectional studies also shown trends or significant association for BMI (p=0.0888), body fat (p=0.0357) and fat mass (p=0.0737), but not for macronutrient intake and energy intake or expenditure (Table 3). Genotypic frequency differences between subjects characterized by a low and a high level of disinhibition or susceptibility to hunger was also addressed (Table 4). The A/A genotypic frequency was higher in the high compared to low disinhibition (0.08 vs 0.17, p=0.0381) and susceptibility to hunger (0.07 vs 0.15, p=0.0154) groups (Table 4).

TABLE 2
Summary of the promising QTLs associated with cognitive restraint, disinhibition
and susceptibility to hunger.

| | | | | SAGE | | |
|---|---|---|---|---|---|---|
| | | | Position | | (LOD | QTDT |
| ^{a}Phenotypes | Chr | Markers | (Mb) | (p-value) | score) | (LOD scores) |
| Restraint | 1p31 | LEPRCA | 65.5 | 0.1412 | 0.25 | 1.47 |
| | 9q22 | D9S938 | 95.2 | 0.2758 | 0.08 | 1.62 |
| | 15q24-q25 | D15S206 | 75.4 | 0.0058 | 1.38 | 1.39 |
| | 17q23-q24 | D17S1351 | 73.7 | 0.1797 | 0.18 | 1.35 |
| | 19p13 | D19S215 | 23.0 | 0.0020 | 1.80 | 0.61 |
| Hunger | 5q31 | D5S1480 | 158.7 | 0.0071 | 1.31 | 0.92 |
| | 13q32 | D13S793 | 97.0 | 0.0049 | 1.45 | 0.36 |
| | 15q21 | LHNLAIII | 54.9 | 0.0022 | 1.76 | 1.03 |
| | 15q24-q25 | D15S206 and | 75.4 | 0.0001 | 3.00 | 1.44 |
| | | D15S171 | 81.8 | 0.0074 | 1.29 | 0.69 |
| | 17q23-q24 | D17S1306, | 55.8 | 0.0061 | 1.36 | 2.06 |
| | | D17S1290 and | 58.8 | 0.0073 | 1.30 | 2.45 |
| | | D17S1351 | 73.7 | 0.0023 | 1.75 | 0.95 |
| | 21q11 | D21S1437 | 18.3 | 0.0111 | 1.14 | 1.42 |

P-values and LOD scores are for age and gender adjusted phenotypes. Loci provided promising evidence of linkage are indicated in bold.
^{a}No significant evidence of linkage was observed for cognitive restraint. Chr = chromosome, Mb = megabases. These linkage analyses were performed on 660 subjects from 202 families.

TABLE 3
Association of the c.217 C > A or p.P73T neuromedin β polymorphism with eating
behaviors and adiposity-related phenotypes in subjects from the QFS.

| | c.217C > A (p.P73T) | | | | |
|---|---|---|---|---|---|
| | A/A^{a} | C/A^{a} | C/C^{a} | | |
| Phenotypes | n = 67 | n = 254 | n = 335 | p-value^{a} | p-value^{b} |
| Cognitive dietary restraint | 6.8 ± 0.53 | 7.2 ± 0.31 | 6.9 ± 0.41 | 0.6102 | 0.6581 |
| Disinhibition | 5.2 ± 0.41^{2,3} | 3.9 ± 0.21 | 4.3 ± 0.30 | 0.0265 | 0.0057 |
| Susceptibility to hunger | 4.8 ± 0.51^{2,3} | 3.5 ± 0.31 | 3.9 ± 0.38 | 0.0343 | 0.0345 |
| Weight (kg) | 67.0 ± 2.7 | 64.9 ± 1.6 | 68.5 ± 2.2 | 0.1067 | — |
| BMI (kg/m²) | 23.8 ± 0.97 | 23.1 ± 0.59³ | 24.6 ± 0.86 | 0.0888 | — |
| Waist girth (cm) | 80.8 ± 2.3 | 79.9 ± 1.5 | 81.9 ± 1.9 | 0.3977 | 0.2930 |
| Body fat (%) | 28.7 ± 1.27 | 26.8 ± 0.87³ | 28.6 ± 0.91 | 0.0357 | — |
| Fat mass (kg) | 21.0 ± 18 | 19.6 ± 1.2³ | 22.2 ± 1.3 | 0.0737 | — |
| Carbohydrate intake (%) | 48.1 ± 1.3 | 49.2 ± 1.1 | 49.6 ± 0.9 | 0.3680 | 0.2556 |
| Protein intake (%) | 16.3 ± 0.7 | 15.6 ± 0.4 | 15.8 ± 0.3 | 0.6366 | 0.6593 |
| Lipid intake (%) | 34. ± 1.2 | 33.6 ± 1.1 | 33.6 ± 1.0 | 0.5613 | 0.5468 |

¹Significantly different when compared to A/A.
²Significantly different when compared to C/A.
³Significantly different when compared to C/C.
ANCOVA (Fisher's LSD):
^{a}Age and gender adjusted phenotypes and
^{b}Age, gender and BMI adjusted phenotypes.
n = maximal number of subjects;
Tabled values represent lsmeans ± SE.

TABLE 4
Genotypic frequencies difference between low and high scores of disinhibition and
hunger for c.217 C > A (p.P73T) neuromedin β polymorphism.

| | Disinhibition | | | Susceptibility to hunger | | |
|---|---|---|---|---|---|---|
| Genotype | Low | High | Odds ratio (95% CI) | Low | High | Odds ratio (95% CI) |
| n | 266 | 102 | | 257 | 145 | |
| | x² = 9.39 | | | x² = 13.03 | | |
| | (p = 0.0091) | | | (p = 0.0015) | | |
| C/C | 0.51 | 0.55 | — | 0.53 | 0.53 | — |
| C/A | 0.41 | 0.28 | 0.7 (0.4-1.1), | 0.41 | 0.32 | 0.8 (0.51-1.23), |
| | | | p = 0.1154^{a} | | | p = 0.2962^{a} |
| A/A | 0.08 | 0.17 | 2.1 (1.04-4.33), | 0.07 | 0.15 | 2.3 (1.18-4.65), |
| | | | p = 0.0381^{b} | | | p = 0.0154^{b} |

Note:
^{a}compared to homozygotes;
^{b}compared to P carriers.
For disinhibition, in men cutoff values were 3 and 8 (0-3, 4-7 and 8-16), while in women the corresponding cutoffs were 4 and 10 (0-4, 5-9 and 10-16). For susceptibility to hunger, cutoff values of 3 and 6 for men and women (0-2, 3-6 and 4-14).

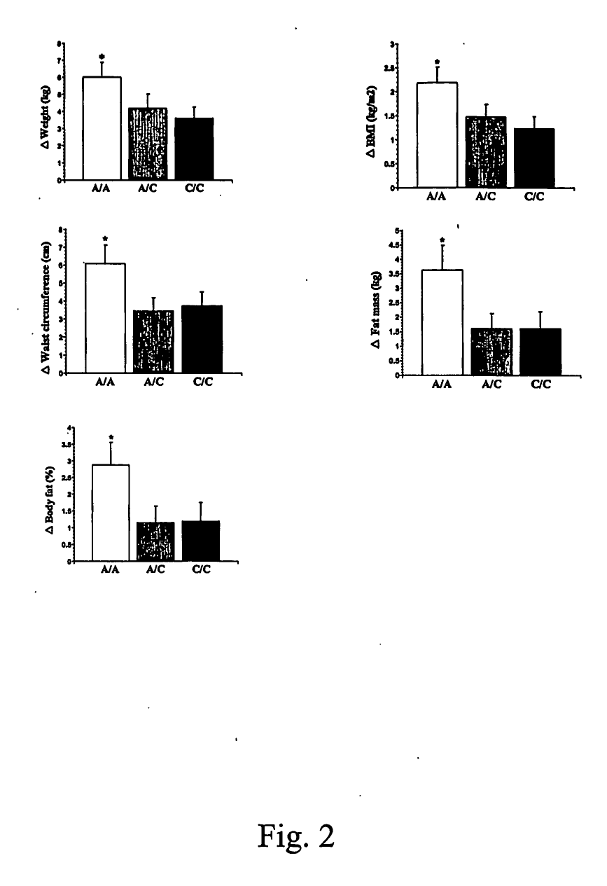

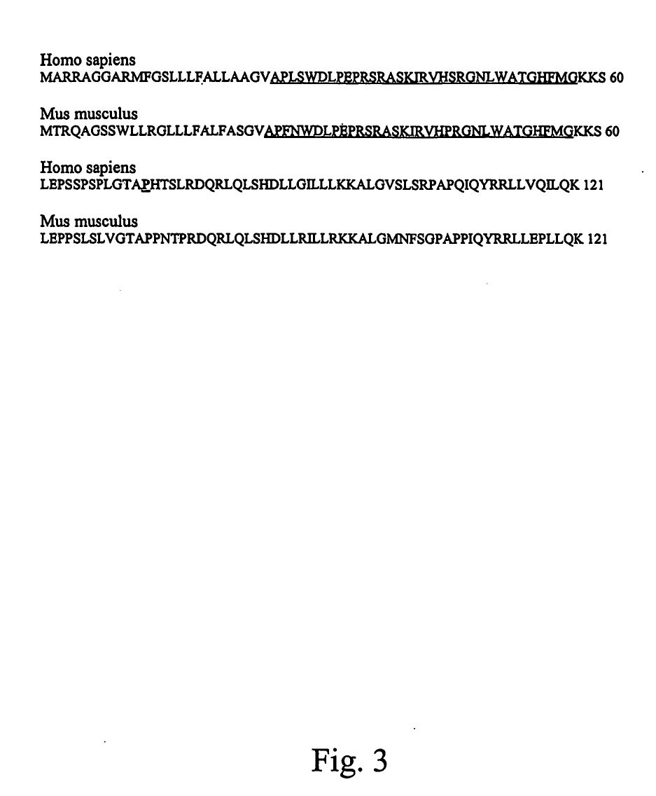

Moreover, significant associations were found between c.217 C>A (the p.P73T) variant and six years changes in adiposity-related phenotypes (Table 5 and FIG. 2). Again, the A/A homozygotes showed about two times more increase, over time, of weight (p=0.03), BMI (p=0.04), waist girth (p=0.02), body fat (p=0.02) and fat mass (p=0.04), compared to the C carriers (Table 5 and FIG. 2). The NMB genotype did not influence changes in energy intake or expenditure but the A/A homozygotes had a tendency to change their macronutrient diet. After the six year follow-up, they ate less protein (p=0.08) and more lipid (p=0.06) when expressed in percent of total energy intake.

TABLE 5
Adiposity and macronutrient intake change between Phase 1 and 2 (6-years follow-
up) for c.217 C > A (p.P73T) neuromedin β polymorphism.

| | A/A | A/C | C/C | p-value |
|---|---|---|---|---|
| n | 26 | 101 | 164 | |
| Weight (kg) | 6.02 ± 0.87^{2,3} | 4.21 ± .082 | 3.63 ± 0.63 | 0.0284 |
| BMI (kg/m²) | 2.19 ± 0.35^{2,3} | 1.47 ± 0.26 | 1.23 ± 0.23 | 0.0365 |
| Waist girth (cm) | 6.10 ± 1.04^{2,3} | 3.44 ± 0.73 | 3.74 ± 0.78 | 0.0183 |
| Body fat (%) | 2.88 ± 0.68^{2,3} | 1.16 ± 0.49 | 1.20 ± 0.56 | 0.0165 |
| Fat mass (kg) | 3.62 ± 0.87^{2,3} | 1.61 ± 0.51 | 1.61 ± 0.58 | 0.0428 |
| Total energy intake (kj)* | 604 ± 664 | 741 ± 393 | 527 ± 363 | 0.7940 |
| Carbohydrate intake (%) | 1.11 ± 1.6 | 1.74 ± 0.9 | 0.74 ± 1.0 | 0.5612 |
| Protein intake (%) | −1.75 ± 0.6^{2,3} | −0.23 ± 0.6 | −0.19 ± 0.5 | 0.0751 |
| Lipid intake (%) | 0.44 ± 1.12² | −1.56 ± 1.12 | 0.10 ± 1.10² | 0.0592 |

*The number of subjects was 15, 40 and 60 for genotypes A/A, A/C and C/C respectively.
Legend:
kg = kilograms,
m2 = squared meter,
cm = centimeter,
kj = kilojoules.

Gastric Neuromedin β Messenger mRNA Levels

The effect of the mutation on neuromedin-β gastric levels was not significant. However, as shown in FIG. 6, the A/A homozygotes tends to have about 16.5% less NMB mRNA as compared to the C carriers (70.3±11.1 vs 86.5±8.5; p=0.16). The neuromedin β c.217 C>A (p.P73T) polymorphism could explains as much as 7% of the variance of the gastric NMB MRNA levels.

### Conclusion

In human, neuromedin-β is a new endocrine factor that should be considered very important in the field of obesity research and eating behaviors and our findings suggest that the neuromedin β is a strong candidate gene for eating behaviors and obesity. A genome-wide linkage analysis led to the identification of four chromosomal regions affecting eating behaviors. The best positional candidate gene, neuromedin-β, was located 0.4 Mb from the linkage peak on chromosome 15q24-q25. A missense mutation located in exon 2 of the neuromedin-β gene was genotyped and found to be associated with disinhibition and susceptibility to hunger as well as changes in body fatness over time. This mutation was also associated with neuromedin-β gastric messenger RNA levels suggesting that neuromedin β gene expression or messenger RNA stability is compromised by the c.217 C>a (p.P73T) substitution. Altogether these results are in accordance with the anorectic effect of neuromedin-β and suggest that in the presence of a lower neuromedin-β MRNA levels, the subjects should have blunted satiety signals, increased disinhibition and susceptibility to hunger and ultimately gain more weight.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. A method for determining susceptibility or predisposition of a patient to obesity comprising identifying in said patient an amino acid substitution in the neuromedin-β polypeptide or a nucleotide substitution in correspondent encoding gene thereof or quantifying said nucleotide sequence level, wherein substitution of at least one nucleotide sequences in said amino acid or nucleotide sequence or variation of nucleotide sequence level compared to a normal patient, is representative of the predisposition or susceptibility to obesity.

2. The method of claim 1, wherein said nucleotide substitution is replacement of a cytosine by an adenine at position 217 of SEQ ID NO:1.

3. The method of claim 1, wherein said amino acid substitution is replacement of proline by a threonine at position 73 of SEQ ID NO:2.

4. The method of claim 1, wherein said obesity is body fatness, abdominal obesity, or visceral obesity.

5. The method of claim 1, wherein said susceptibility or predisposition of a patient to obesity is representative of disinhibition or susceptibility or predisposition to hunger.

6. A method for diagnosing predisposition or susceptibility to neuromedin-β associated eating behavior disorder comprising the steps of:
a) characterizing sequence or quantity of nucleotide encoding for neuromedin-β or amino acid sequence of neuromedin-β in a biologic sample of a patient; and
b) determining the presence or absence of nucleic or amino acid substitution or the quantity of said nucleic acid sequence in said characterized biological sample of step a);
wherein substitution of at least one nucleotide or amino acid in said nucleotide or amino acid sequence, or variation of quantity of said nucleotide compared to a normal patient, is representative of the predisposition or susceptibility to eating disorders.

7. The method of claim 6, wherein said nucleotide sequence is DNA or a RNA.

8. The method of claim 6, wherein said eating disorder is cognitive dietary restraint, disinhibition and susceptibility to hunger, known characteristics of binge eating disorders, bulimia nervosa, or anorexia nervosa.

9. The method of claim 6, wherein said substitution is corresponding to the substitution of the cytosine by an adenine at position 217 of SEQ ID NO:1.

10. The method of claim 6, wherein said substitution is corresponding to the substitution of a proline by a threonine at position 73 of SEQ ID NO:2.

11. The method of claim 6, wherein said quantity gastric neuromedin , messenger RNA levels?


SEQUENCE LISTING (35 sequences)

SEQ ID NO: 1 - DNA, 654 bp, Artificial Sequence
  Synthetic Neuromedin cDNA
ctgttacccgggaggagagctcctcgcccgacctctaccctcatgaagagaggctcagagttaccttaggcgagacttaaccgaatcttctaaccgctggtgtgtttttgctgcacctcggaaaagctgagggagcaggctttgccaccacccagacacctttgtggctccttggtgaccagcccatccccattggggacagctccccacacctccctgagggaccagcgactgcagggccatccccggatcctgcatgggaggaattaccacccagtactgtattagggtgtgacgcagagctcaaaggaggaacagtccaaagaaaggaagctgaccttcccagtagaccccatgtgaggacgctgacactagcccagcaccaagcactgtatttggattttcttccacgatcaatggcaggatgcccctatctttatcaggagcccctccctggctcaattcttctgtatgtaatggggcagacacaacagcgtggcttagattgtgcccacccagggaaggtgctgaatggtgctgaatgggaccctgttgatggccccatctggatgtaaatcctgagctcaaatctctataaaaccttgctctttacatacaatgcctggtcctctcctttcacccgtcttttagggg

SEQ ID NO: 2 - protein, 121 aa, Artificial Sequence
  Synthetic peptide
MARRAGGARMFGSLLLFALLAAGVAPLSWDLPEPRSRASKIRVHSRGNLWATGHFMGKKSLEPSSPSPLGTAPHTSLRDQRLQLSHDLLGILLLKKALGVSLSRPAPQIQYRRLLVQILQK

SEQ ID NO: 3 - DNA, 18 bp, Artificial Sequence
  Neuromedin forward PCR primer
tgcagtcgctggtccctc

SEQ ID NO: 4 - DNA, 20 bp, Artificial sequence
  Neuromedin reverse PCR primer
aggcgagacttaaccgaatc

SEQ ID NO: 5 - DNA, 17 bp, Artificial Sequence
  Neuromedin mini-sequencing primer
cctcagggaggtgtggg

SEQ ID NO: 6 - DNA, 19 bp, Artificial Sequence
  Neuromedin mRNA quantification forward primer
ttccagcccatccccattg

SEQ ID NO: 7 - DNA, 21 bp, Artificial Sequence
  Neuromedin mRNA quantification reverse primer
caacagggaagcaggaaatac

SEQ ID NO: 8 - DNA, 20 bp, Artificial Sequence
  L27 mRNA quantification forward primer
gggcaagttcatgaaacctg

SEQ ID NO: 9 - DNA, 20 bp, Artificial Sequence
  L27 mRNA quantification reverse primer
ccttgtgggcattaggtgat

SEQ ID NO: 10 - protein, 121 aa, Homo sapiens; identical to SEQ ID NO: 2
  neuromedin-beta (1)..(121)

SEQ ID NO: 11 - protein, 121 aa, Mus musculus
  neuromedin-beta (1)..(121)
MTRQAGSSWLLRGLLLFALFASGVAPFNWDLPEPRSRASKIRVHPRGNLWATGHFMGKKSLEPPSLSLVGTAPPNTPRDQRLQLSHDLLRILLRKKALGMNFSGPAPPIQYRRLLEPLLQK

SEQ ID NO: 12 - DNA, 3677 bp, Homo sapiens
  neuromedin-beta (1)..(3677)
ctgttacccgggaggagagctcctcgcccgacctctaccctcatgaagagaggctcagagggctgaagtgcctatttggccgaaagccgtggcagagtggcaaggcagggccaggggaagcggctccgccgccggggccgggcccctgtttggccggtgcccggtccttagcctgaaggtggcgggcttccgccagaagcccctggcggaagcggtgcccgcgtgcgggccagagtgtgggtgtgcaggtctctgggcggcccaaagggggtgcccctgcctggtaacctagcgggagggtggggacggcggggagggcggcgggcgcggggcacggctccgctgctcagggcaggctccgcccccaggggcgcggatttaaaaggatcgaaggcagccccggagcccagcggccgggaagcgcgcccgaacgaagccgcggcccgggcacagccatggcccggcgggcggggggcgctcggatgttcggcagcctcctgctcttcgccctgctcgctgccggcgtcgccccgctcagctgggatctcccggagccccgcagccgagccagcaagatccgagtgcactcgcgaggcaacctctgggccaccggtaagtctttggggacggagcaagcaagcgcccctcatccagttcagaccccatttccttctcaaccctctggccgctcctcagccacggacactagtgtcggagcaggtggaaaaccctggggctcatctaatttaatagatatgtacttgagacccggacaggtcagtgacttggctaaggtcgcgcagccagtttaagacagggctgggctagatcctaaatcccactgccagccggtgccccttaccttaggcgagacttaaccgaatcttctaaccgctggtgtgtttttgctgcacctccactttccaggcgcctcttcactctccacttcctaccctgccctttttcgtcccttgtccaagcagcccacacaactagcagagtttctccctggccctggaccatcccaccttcctgccagctgtgccatcctctctacctgttcaggaaaagctgagggagcaggctttgccaccacccagacacctttgtggctccttggtgaggtggaagcaccaagaggaggaaggttaagtgtcttcccgctacaagaacggaaacgtgggagagatgaggaacttttcctctgaggtaggatcctggctgcttgacttccttgtgcctggacacctcctttccaggtcacttcatgggcaagaagagtctggagccttccagcccatccccattggggacagctccccacacctccctgagggaccagcgactgcagctgagtcatgatctgctcggaatcctcctgctaaagaaggctctgggcgtgagcctcagccgccccgcaccccaaatccaggtgagccgggcccctgctccaatgtcaggagggcccagctggggccatccccggatcctgcatgggaggaattaccacccagtactgtattagggtgtgactgtctgactaggacattatgggtgtggaccccagaaagccaggtttccaggcttttccctcttgaggcagagctcaaaggaggaacagtccaaagaaaggaagctgaccttcccagtagaccccatggggcaagaagtagggaaagaagttcccctgactcatcacccagtctaaagtaacagactgggatcatcagccttttggagcaggacctttctcccccagtctacacagtcttgtcccacccatgctgttccctgctgcattagtcaagtccacctttgctgtgtcctgtgtatgcttgtggccggaatgggaccctgaggcccagagagggaaagagaagtcagcagccaagacagagtctggaccttgttcacctggactggagctcttcccattctctcatctgcctcagtatccagtggtagggtttagcaacttcagtacatttgacgttctaggctgaatcactgtttcttgtgagggctgacgtgtgcatcatgggatgtttggcagcatccctggtctctacatactagatgccagtagcatctttcccccccatcaagttgtgacaactgaaaagacctccagacatcaccagatgtctgctgggggagagggccccaaatcattattggttgtcagtcactgatctatgggattcaagactccaaagctggagccaagctagcctcaaggtatagccccgctgagtggcaactccttcattccctgctcctggtgctgtccttactgcacaccaccctccctggtcctgctgctgtgtgcagaaggcagtgtgatgtggtagaatgtgggttttgactacaacgtgctgggctcataacctagctacttagtagctgtatgaccttagaaatgtcccttaacttctctaaagcctcaatattcttcacccataaaatgaagataataaggcccatctcccattaaatgagaccatttatgtcaaatgctcagcatggtgcctggctcatagacagcccttagtagatgcgagctcttatcagtctgtgagctccctggcggcacctgttgtagactcgccttcatatcccccagtgtgcctagcatatagtgtgtgcattttgaagggagaggcattccctagaaaaggtccaacccagcctcaaccaacatccctgacttcctgaggcacagaaccagcagcccctgaggacctcagatgtaaggcctaggagcttgggctggctgagtctgaagggaaacaatgtcacctctaatgcccttggttttgaagctctgacacatgcagaccaactagagaatctcagaagcagcagtgcctacgtctggggcttcagagtgaggtctggggcagagctggggtgggggagtgaggacgctgacactagcccagcaccaagcactgtatttggattttcttccacgatcttctttgactgtcatgaccaccctgggtggttggggctgtgttctgagaaaactactgccccgacccctccaaggcaattcagggtgctctggggcctgcctcagctgacagcctgctggtgcccacctctgcagacatctggcacttaggaatggcaggatgcccctatctttatcaggagcccctccctggctcaattcttctgtatgtttctcttcagtacaggaggctgctggtacaaatactgcagaaatgacaccaataatggggcagacacaacagcgtggcttagattgtgcccacccagggaaggtgctgaatgggaccctgttgatggccccatctggatgtaaatcctgagctcaaatctctgttactccattactgtgatttctggctgggtcaccagaaatatcgctgatgcagacacagattatgttcctgctgtatttcctgcttccctgttgaattggtgaataaaaccttgctctttacatacaatgcctggtcctctcctttcacccgtcttttaggggatgggaggaaaagggggctggagggcagagtgttcagtgagatggggctggctcaaaaagtccagaataccccatgccatggcaccagcctggcactgactctggcctcctacccattcattcagcaagcaaataacgcctgttgcatgccaggcttcgtgcc

SEQ ID NO: 13 - DNA, 3677 bp, Homo sapiens
  Neuromedin-beta_mutant (1)..(3677)
ctgttacccgggaggagagctcttcgcccgacctctaccctcatgaagagaggctcagagggctgaagtgcctatttggccgaaagccgtggcagagtggcaaggcagggccaggggaagcggctccgccgccggggccgggcccctgtttggccggtgcccggtccttagcctgaaggtggcgggcttccgccagaagcccctggcggaagcggtgcccgcgtgcgggccagagtgtgggtgtgcaggtctctgggcggcccaaagggggtgcccctgcctggtaacctagcgggagggtggggacggcggggagggcggcgggcgcggggcacggctccgctgctcagggcaggctccgcccccaggggcgcggatttaaaaggatcgaaggcagccccggagcccagcggccgggaagcgcgcccgaacgaagccgcggcccgggcacagccatggcccggcgggcggggggcgctcggatgttcggcagcctcctgctcttcgccctgctcgctgccggcgtcgccccgctcagctgggatctcccggagccccgcagccgagccagcaagatccgagtgcactcgcgaggcaacctctgggccaccggtaagtctttggggacggagcaagcaagcgcccctcatccagttcagaccccatttccttctcaaccctctggccgctcctcagccacggacactagtgtcggagcaggtggaaaaccctggggctcatctaatttaatagatatgtacttgagacccggacaggtcagtgacttggctaaggtcgcgcagccagtttaagacagggctgggctagatcctaaatcccactgccagccggtgccccttaccttaggcgagacttaaccgaatcttctaacggctggtgtgtttttgctgcacctccactttccaggcgcctcttcactctccacttcctaccctgccctttttcgtcccttgtccaagcagcccacacaactagcagagtttctccctggccctggaccatcccaccttcctgccagctgtgccatcctctctacctgttcaggaaaagctgagggagcaggatttgccaccacccagacacctttgtggctccttggtgaggtggaagcaccaagaggaggaaggttaagtgtcttcccgctacaagaacggaaacgtgggagagatgaggaacttttcctctgaggtaggatcctggctgcttgacttccttgtgcctggacacctcctttccaggtcacttcatgggcaagaagagtctggagccttccagcccatccccattggggacagctacccacacctccctgagggaccagcgactgcagctgagtcatgatctgctcggaatcctcctgctaaagaaggctctgggcgtgagcctcagccgccccgcaccccaaatccaggtgagccgggcccctgctccaatgtcaggagggcccagctggggccatccccggatcctgcatggaaggaattaccacccagtactgtattagggtgtgactgtctgactaggacattatgggtgtggaccccagaaagccaggtttccaggcttttccctcttgaggcagagctcaaaggaggaacagtccaaacaaaggaagctgaccttcccagtagaccccatggggcaagaagtagggaaagaagttcccctgactcatcacccagtctaaagtaacagactgggatcatcagccttttggagcaggacctttctcccccagtctacacagtcttgtcccacccatgctgttccctgctgcattagtcaagtccacctttgctgtgtcctgtgtatgcttgtggccggaatgggaccctgaggcccagagagggaaagagaagtcagcagccaagacagagtctggaccttgttcacctggactggagctcttcccattctctcatctgcctcagtatccagtggtagggtttagcaacttcagtacatttgacgttctaggctgaatcactgtttcttgtgagggctgacgtgtgcatcatgggatgtttggcagcatccctggtctctacatactagatgccagtagcatctttcccccccatcaagttgtgacaactgaaaagacctccagacatcaccagatgtctgctgggggagagggccccaaatcattattggttgtcagtcactgatctatgggattcaagactccaaagctggagccaagctagcctcaaggtatagccccgctgagtggcaactccttcattccctgctcctggtgctgtccttactgcacaccaccctccctggtcctgctgctgtgtgcagaaggcagtgtgatgtggtagaatgtgggttttgactacaacgtgctgggctcataacctagctacttagtagctgtatgaccttagaaatgtcccttaacttctctaaagcctcaatattcttcacccataaaatgaagataataaggcccatctcccattaaatgagaccatttatgtcaaatgctcagcatggtgcctggctcatagacagcccttagtagatgcgagctcttatcagtctgtgagctccctggcggcacctgttgtagactcgccttcatatcccccagtgtgcctagcatatagtgtgtgcattttgaagggagaggcattccctagaaaaggtccaacccagcctcaaccaacatccctgacttcctgaggcacagaaccagcagcccctgaggacctcagatgtaaggcctaggagcttgggctggctgagtctgaagggaaacaatgtcacctctaatgcccttggttttgaagctctgacacatgcagaccaactagagaatctcagaagcagcagtgcctacgtctggggcttcagagtgaggtctggggcagagctggggtgggggagtgaggacgctgacactagcccagcaccaggcactgtatttggattttcttccacgatcttctttgactgtcatgaccaccctgggtggttggggctgtgttctgagaaaactactgccccgacccctccaaggcaattcagggtgctctggggcctgcctcagctgacagcctgctggtgcccacctctgcagacatctggcacttaggaatggcaggatgcccctatctttatcaggagcccttccctggctcaattcttctgtatgtttctcttcagtacaggaggctgctggtacaaatactgcagaaatgacaccaataatggggcagacacaacagcgtggcttagactgtgcccacccagggaaggtgctgaatgggaccctgttgatggccgcatctggatgtaaatcctgagctcaaatctctgttactccattactgtgatttctggctgggtcaccagaaatatcgctgatgcagacacagattatgttcctgctgtatttcctgcttccctgttgaattggtgaataaaaccttgctctttacataaaatgcctggtcctctcctttcacccgtcttttaggggatgggaggaaaagggggctggagggcagagtgttcagtgagatggggctggctcaaaaagtccagaataccccatgccatggcaccagcctggcactgactctggcctcctacccattcattcagcaagcaaataacgcctgttgcatgccaggcttcgtgcc

SEQ ID NO: 14 - DNA, 60 bp, Homo sapiens
ctgttacccgggaggagagctcctcgcccgacctctaccctcatgaagagaggctcagag

SEQ ID NO: 15 - DNA, 60 bp, Homo sapiens
ctgttacccgggaggagagctcttcgcccgacctctaccctcatgaagagaggctcagag

SEQ ID NO: 16 - DNA, 60 bp, Homo sapiens
ttaccttaggcgagacttaaccgaatcttctaaccgctggtgtgtttttgctgcacctcc

SEQ ID NO: 17 - DNA, 60 bp, Homo sapiens
ttaccttaggcgagacttaaccgaatcttctaacggctggtgtgtttttgctgcacctcc

SEQ ID NO: 18 - DNA, 60 bp, Homo sapiens
ggaaaagctgagggagcaggctttgccaccacccagacacctttgtggctccttggtgag

SEQ ID NO: 19 - DNA, 60 bp, Homo sapiens
ggaaaagctgagggagcaggatttgccaccacccagacacctttgtggctccttggtgag

SEQ ID NO: 20 - DNA, 60 bp, Homo Sapiens
ccagcccatccccattggggacagctccccacacctccctgagggaccagcgactgcagc

SEQ ID NO: 21 - DNA, 60 bp, Homo Sapiens
ccagcccatccccattggggacagctacccacacctccctgagggaccagcgactgcagc

SEQ ID NO: 22 - DNA, 60 bp, Homo Sapiens
ggccatccccggatcctgcatgggaggaattaccacccagtactgtattagggtgtgact

SEQ ID NO: 23 - DNA, 60 bp, Homo Sapiens
ggccatccccggatcctgcatggaaggaattaccacccagtactgtattagggtgtgact

SEQ ID NO: 24 - DNA, 60 bp, Homo Sapiens
gcagagctcaaaggaggaacagtccaaagaaaggaagctgaccttcccagtagaccccat

SEQ ID NO: 25 - DNA, 60 bp, Homo Sapiens
gcagagctcaaaggaggaacagtccaaacaaaggaagctgaccttcccagtagaccccat

SEQ ID NO: 26 - DNA, 60 bp, Homo Sapiens
gtgaggacgctgacactagcccagcaccaagcactgtatttggattttcttccacgatct

SEQ ID NO: 27 - DNA, 60 bp, Homo Sapiens
gtgaggacgctgacactagcccagcaccaggcactgtatttggattttcttccacgatct

SEQ ID NO: 28 - DNA, 60 bp, Homo Sapiens
aatggcaggatgcccctatctttatcaggagcccctccctggctcaattcttctgtatgt

SEQ ID NO: 29 - DNA, 60 bp, Homo Sapiens
aatggcaggatgcccctatctttatcaggagcccttccctggctcaattcttctgtatgt

SEQ ID NO: 30 - DNA, 60 bp, Homo Sapiens
taatggggcagacacaacagcgtggcttagattgtgcccacccagggaaggtgctgaatg

SEQ ID NO: 31 - DNA, 60 bp, Homo Sapiens
taatggggcagacacaacagcgtggcttagactgtgcccacccagggaaggtgctgaatg

SEQ ID NO: 32 - DNA, 60 bp, Homo Sapiens
gtgctgaatgggaccctgttgatggccccatctggatgtaaatcctgagctcaaatctct

SEQ ID NO: 33 - DNA, 60 bp, Homo Sapiens
gtgctgaatgggaccctgttgatggccgcatctggatgtaaatcctgagctcaaatctct

SEQ ID NO: 34 - DNA, 60 bp, Homo Sapiens
ataaaaccttgctctttacatacaatgcctggtcctctcctttcacccgtcttttagggg

SEQ ID NO: 35 - DNA, 60 bp, Homo Sapiens
ataaaaccttgctctttacataaaatgcctggtcctctcctttcacccgtcttttagggg